(19) United States
(12) Patent Application Publication
(10) Pub. No.: US 2021/0379197 A1
(43) Pub. Date: Dec. 9, 2021

(54) DUAL-TARGETING LIPID-POLYMER HYBRID NANOPARTICLES

(71) Applicant: IUCF-HYU (INDUSTRY-UNIVERSITY COOPERATION FOUNDATION HANYANG UNIVERSITY), Seoul (KR)

(72) Inventors: Yong-Hee KIM, Seoul (KR); Seok-Beom YONG, Seoul (KR); Jee Young CHUNG, Seoul (KR); Seong Su KIM, Seoul (KR); Jae Hyun KIM, Seoul (KR); Se Hee RA, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 17/228,224

(22) Filed: Apr. 12, 2021

(30) Foreign Application Priority Data
Jun. 3, 2020 (KR) 10-2020-0066875
Oct. 30, 2020 (KR) 10-2020-0142949

(51) Int. Cl. A61K 47/68; A61K 9/51; A61K 45/06; A61K 33/30; A61K 33/26; A61K 33/32; A61K 33/24; A61K 47/54; A61K 47/69; A61P 35/02

(52) U.S. Cl. CPC A61K 47/6887; A61K 9/5153; A61K 9/5123; A61K 45/06; A61K 33/30; A61K 33/26; A61P 35/02; A61K 33/24; A61K 47/546; A61K 47/6925; A61K 47/6889; A61K 47/6867; A61K 33/32

(57) ABSTRACT

The present invention relates to dual-targeting lipid-polymer hybrid nanoparticles (T-hNPs) comprising a polymer core comprising a heme oxygenase 1 inhibitor and a lipid membrane (shell) comprising a targeting moiety, a kit for preparing the dual-targeting lipid-polymer hybrid nanoparticles, a pharmaceutical composition comprising the dual-targeting lipid-polymer hybrid nanoparticles as an active ingredient, and a method of preventing or treating cancer comprising administering the pharmaceutical composition to a subject in need thereof. Accordingly, the present invention can provide the dual-targeting lipid-polymer hybrid nanoparticles (T-hNPs) comprising a polymer core comprising a heme oxygenase 1 inhibitor and a lipid membrane (shell) comprising a targeting moiety, the kit for preparing the dual-targeting lipid-polymer hybrid nanoparticles (T-hNPs), the pharmaceutical composition comprising the dual-targeting lipid-polymer hybrid nanoparticles (T-hNPs) as an active ingredient, and the method of preventing or treating cancer comprising administering the pharmaceutical composition to a subject in need thereof.

### CROSS-REFERENCE TO RELATED APPLICATION

This application claims priority to and the benefit of Korean Patent Application No. 10-2020-0066875, filed on Jun. 3, 2020, and Korean Patent Application No. 10-2020-0142949, filed on Oct. 30, 2020, the disclosure of which is incorporated herein by reference in its entirety.

### BACKGROUND

### Technical Field

The present invention relates to dual-targeting lipid-polymer hybrid nanoparticles (T-hNPs) comprising a polymer core comprising a heme oxygenase 1 inhibitor and a lipid membrane (shell) comprising a targeting moiety, a kit for preparing the dual-targeting lipid-polymer hybrid nanoparticles (T-hNPs), a pharmaceutical composition comprising the dual-targeting lipid-polymer hybrid nanoparticles (T-hNPs) as an active ingredient, and a method of preventing and treating cancer comprising administering the pharmaceutical composition to a subject in need thereof.

### Background Art

To date, the main methods of cancer treatment are surgery and radiation treatment as local treatment, and chemotherapy and molecular target therapy as systemic treatment. These treatment methods are applied to patients either alone or as a combination of various therapies depending on the tumor cell type, stage, recurrence, and compliance with the patient's treatment. However, despite various efforts to improve cancer treatment rates, comprising interdisciplinary studies, the improvement in patient survival rates over the past decades has been insignificant. In recent years, as the importance of organ preservation therapy has increased due to concerns about the quality of life of patients, and the view of carcinoma as a systemic disease has emerged in consideration of the effect of distant metastasis on the survival rate, some therapeutic regimens, comprising chemotherapy have been appearing in treatment of cancer.

In the case of existing anti-cancer chemicals, it is difficult to obtain sufficient drug concentration in a tumor to kill cancer cells as the anti-cancer chemical is distributed in the body non-specifically without distinction between normal cells and cancer cells. At the same time, inadequate treatment is made due to toxicity to normal cells. Moreover, in some water-insoluble anti-cancer drugs, it is challenging to administer medications intravenously, and various side effects depending on the organic solvent used to dissolve the drugs are also problematic.

Meanwhile, nanoparticles (NPs) are particles, devices, or systems with a size of less than a micron and can be made of various materials, such as metals, ceramics, polymers, fats, proteins, etc. Because the size of nanoparticles is very small, they can pass through various barriers in the body, such as the blood-brain barrier, and since the ratio of the relative volume to the size is not large, a large amount of drugs can be loaded and delivered. Most of the nanoparticles used in drug delivery systems are water-soluble in order to be administered intravenously, and they do not accumulate in the body and can be excreted through normal metabolic functions.

In addition, lipid-polymer hybrid nanoparticles have been used as a scaffold for fusing the properties of both lipid and polymer nanoparticles in drug delivery systems. The lipid membrane acts as a barrier to protect the drug from excessive leakage, assists in achieving controlled drug release, and the polymer aids in the structural stability of the lipid membrane. Also, studies using molecular probes on the surface of hybrid nanoparticles are being conducted for the delivery of drugs for effective drug targeting. For example, the surface of hybrid nanoparticles can be changed with folic acid, transferrin, monoclonal antibodies, peptides, cytokines, DNA sequences, specific antibodies for immunotherapy, or fragments of antibodies.

As these lipid-polymer hybrid nanoparticles can effectively deliver chemotherapeutic drugs to cancer tissues and release the drugs through specific external or internal stimuli, thereby improving therapeutic effects and minimizing side effects, they are becoming a hot topic in the field of cancer treatment.

### DISCLOSURE

### Technical Problem

The present invention is directed to providing dual-targeting lipid-polymer hybrid nanoparticles (T-hNPs) comprising a polymer core comprising a heme oxygenase 1 inhibitor and a lipid membrane (shell) comprising a targeting moiety.

The present invention is also directed to providing a method of preparing the dual-targeting lipid-polymer hybrid nanoparticles (T-hNPs) according to the present invention comprising adding a heme oxygenase 1 inhibitor and a polymer mixture to an aqueous lipid solution in a dropwise manner and performing sonication to generate lipid-polymer hybrid nanoparticles; and forming a targeting moiety on the lipid-polymer hybrid nanoparticles.

The present invention is also directed to providing a kit for preparing the dual-targeting lipid-polymer hybrid nanoparticles (T-hNPs) according to the present invention comprising a container comprising a targeting moiety and a container comprising lipid-polymer hybrid nanoparticles.

The present invention is also directed to providing a pharmaceutical composition comprising the dual-targeting lipid-polymer hybrid nanoparticles (T-hNPs) according to the present invention as an active ingredient.

The present invention is also directed to providing a method of preventing or treating cancer comprising administering the pharmaceutical composition according to the present invention to a subject in need thereof.

### Technical Solution

In order to achieve the above objects, the present invention provides dual-targeting lipid-polymer hybrid nanoparticles (T-hNPs) comprising a polymer core comprising a heme oxygenase 1 inhibitor and a lipid membrane (shell) comprising a targeting moiety.

Also, the present invention provides a method of preparing the dual-targeting lipid-polymer hybrid nanoparticles (T-hNPs) according to the present invention comprising adding a heme oxygenase 1 inhibitor and a polymer mixture to an aqueous lipid solution in a dropwise manner and performing sonication to generate lipid-polymer hybrid nanoparticles, and forming a targeting moiety on the lipid-polymer hybrid nanoparticles.

Also, the present invention provides a kit for preparing the dual-targeting lipid-polymer hybrid nanoparticles (T-hNPs) according to the present invention comprising a container comprising a targeting moiety and a container comprising lipid-polymer hybrid nanoparticles.

Also, the present invention provides a pharmaceutical composition comprising the dual-targeting lipid-polymer hybrid nanoparticles (T-hNPs) as an active ingredient.

Also, the present invention provides a method of preventing or treating cancer comprising administering the pharmaceutical composition according to the present invention to a subject in need thereof.

Hereinafter, the configuration of the present invention will be described in detail. The present invention provides dual-targeting lipid-polymer hybrid nanoparticles (T-hNPs) comprising a polymer core comprising a heme oxygenase 1 inhibitor and a lipid membrane (shell) comprising a targeting moiety.

The heme oxygenase 1 (HO1) is an antioxidant, heme-degrading enzyme known to have a cytoprotective function in various tissues, and such an HO1 inhibitor is used as an anti-cancer immune checkpoint inhibitor.

The anti-cancer immune checkpoint inhibitors are also known as immune checkpoint inhibitors or anti-cancer immune agents. When the immune checkpoint protein, which is a checkpoint possessed by a cancer cell, binds to a T cell, the function of the T cell is lost.

To this day, the FDA has approved some products as anti-cancer checkpoint inhibitors that neutralize the immune checkpoint protein of cancer cells. For example, Atezolizumab (brand name: Tecentriq) is efficacious for treatment of non-small cell lung cancer, urinary tract cancer, small cell lung cancer, and triple-negative breast cancer; duvalumab (brand name:Imfinzi) is efficacious for treatment of bladder cancer and non-small cell lung cancer; ipilimumab (brand name: Yervoy) is efficacious for treatment of melanoma, and renal cell carcinoma (kidney cancer); pembrolizumab (brand name: Keytruda) is efficacious for treatment of melanoma, non-small cell lung cancer, renal cell carcinoma, Hodgkin lymphoma, head and neck cancer, and urinary tract carcinoma; and nivolumab (brand name: Opdivo) is efficacious for treatment of melanoma, non-small cell lung cancer, renal cell cancer, Hodgkin lymphoma, head and neck cancer, and stomach cancer.

In the present invention, in addition to the heme oxygenase 1 inhibitor, any anti-cancer immune checkpoint inhibitor (or immune checkpoint inhibitor) may be used without limitation as long as it is an anti-cancer checkpoint inhibitor known in the art. For example, CTLA-4 inhibitors, PD-1 inhibitors, and PD-L1 inhibitors can be used.

In the present invention, the dual-targeting lipid-polymer hybrid nanoparticles of the present invention comprise a lipid membrane containing a targeting moiety. The lipid membrane (shell) acts as a barrier to protect the drug from excessive leakage in the nanoparticles, helps achieve controlled drug release, and increases influx to target cells.

In one embodiment, the lipid constituting the lipid membrane of the present invention may comprise one or more selected from among distearoylphosphatidylethanolamine (DSPE-PEG2000), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-phosphocholine (DOPC), 1,2-dimyristoyl-sn-glycero-3-phosphoethanolamine (DMPE), 1,2-dipalmitoyl-sn-glycero-3-phosphoethanolamine (DPPE), 1,2-dioleoyl-sn-glycero-3-phosphoethanolamine (DOPE), 1,2-dimyristoyl-sn-glycero-3-phosphate (DMPA-Na), 1,2-dipalmitoyl-sn-glycero-3-phosphate (DPPA-Na), 1,2-dioleoyl-sn-glycero-3-phosphate (DOPA-Na), 1,2-dimyristoyl-sn glycero-3-phosphoglycerol (DMP G-Na), 1,2-dipalmitoyl-sn-glycero-3-phosphoglycerol (DPPG-Na), 1,2-dioleoyl-sn-glycero-3-phosphoglycerol (DOPG-Na), 1,2-dimyristoyl-sn-glycero-3-phosphoserine (DMPS-Na), 1,2-dipalmitoyl-sn-glycero-3-phosphoserine (DPPS-Na), 1,2-dioleoylsn-glycero-3-phosphoserine (DOPS-Na), 1,2-dioleoyl-sn-glycero-3-phosphoethanolamine (DOPE-Glutaryl-(Na)2), tetramyristoyl cardiolipin-(Na)2, 1,2-distearoyl-sn-glycero-3-phosphoethanolamine (DSPE-mPEG-2000-Na), DSPE-mPEG-5000-Na, DSPE-maleimide PEG-2000-Na, 1,2-dioleoyl-3-trimethylammonium-propane (DOTAP-C1), and combinations thereof, but the present invention is not limited thereto.

In one embodiment, the targeting moiety is immobilized on the lipid membrane of the present invention through a non-covalent bond.

In one embodiment, the non-covalent bond comprises a biotin/avidin or biotin/streptavidin bond.

Specifically, the biotin may be bound to one end of the lipid, and the avidin or streptavidin may be bound to one end of the targeting moiety, but the present invention is not limited thereto.

Also, the dual-targeting lipid-polymer hybrid nanoparticles (T-hNPs) of the present invention comprise a polymer core comprising a heme oxygenase 1 inhibitor.

As described above, the heme oxygenase 1 inhibitor may be used as an anti-cancer immune checkpoint inhibitor (or immune checkpoint inhibitor).

In one embodiment, the heme oxygenase 1 inhibitor may be selected from the group consisting of SnMP, ZnMP, FeMP, MnMP, CrMP, SnPP, CrPP, and MnPP, but is not limited thereto.

The polymer core containing such a heme oxygenase 1 inhibitor plays a role in helping the structural stability of the lipid membrane of the nanoparticles, the polymer core is used for loading poorly soluble/insoluble drugs into the nanoparticles, and the heme oxygenase 1 inhibitor may be loaded into the polymer core.

In one embodiment, the heme oxygenase 1 inhibitor is loaded into the polymer core in an amount capable of reaching the target cell and exerting an effective effect, preferably 3 to 7 w/w %, and more preferably, 4 to 6.3 w/w % based on the mass of the polymer.

In the present invention, the polymer comprised in the dual-targeting lipid-polymer hybrid nanoparticles (T-hNPs) is not largely limited as long as it is a biocompatible polymer used in the art, but the polymer may comprise one or more selected from the group consisting of poly(L-lactide) (PLLA), polyglycolic acid (PGA), polylactic acid (PLA), poly(lactic-co-glycolic acid), polycaprolactone (PCL), and poly (3-hydroxybutyrate-co-3-hydroxyvalerate) (PHBV), or a copolymer thereof.

In one embodiment, the lipid membrane and the polymer core may be mixed so that the content of the lipid membrane is 0.15 to 0.35 w/w %, and preferably 0.20 to 0.30 w/w %, based on the mass of the polymer core.

In one embodiment, the dual-targeting lipid-polymer hybrid nanoparticles (T-hNPs) of the present invention are prepared by preparing a polymer mixture by mixing DSPE-PEG2000 (biotinylated: non-biotinylated) and DPPC in a molar ratio of 1:1 to 5, adding the polymer mixture to an aqueous lipid solution in a dropwise manner, mixing, and sonicating to generate lipid-polymer hybrid nanoparticles (hNPs) and then forming a targeting moiety on the lipid-polymer hybrid nanoparticles to prepare the dual-targeting lipid-polymer hybrid nanoparticles (T-hNPs).

Accordingly, the present invention provides a method of preparing the dual-targeting lipid-polymer hybrid nanoparticles (T-hNPs) comprising adding the heme oxygenase 1 inhibitor and the polymer mixture to an aqueous lipid solution in a dropwise manner and performing sonication to generate the lipid-polymer hybrid nanoparticles, and forming a targeting moiety on the lipid-polymer hybrid nanoparticles.

Meanwhile, the present invention provides a kit for producing the dual-targeting lipid-polymer hybrid nanoparticles (T-hNPs) according to the present invention comprising a container comprising a targeting moiety and a container comprising lipid-polymer hybrid nanoparticles.

In the present invention, the kit comprises one or more containers comprising the composition described in the present invention, and if necessary, may comprise instructions associated with the packaging in the form directed by the government agency overseeing the manufacture, use and sale of the drug. The kit may further comprise a description of the selection or treatment of a suitable individual.

Also, the present invention provides a pharmaceutical composition comprising the dual-targeting lipid-polymer hybrid nanoparticles (T-hNPs) as an active ingredient.

In the pharmaceutical composition, the dual-targeting lipid-polymer hybrid nanoparticle (T-hNPs) may be in a lyophilized form.

In the present invention, the lipid-polymer hybrid nanoparticles (T-hNPs) may have a diameter of 150 to 200 nm.

In the pharmaceutical composition, all the above-described contents may be applied as they are to the dual-targeting lipid-polymer hybrid nanoparticle (T-hNPs).

The pharmaceutical composition of the present invention may contain a pharmaceutically acceptable carrier. In the present invention, the term “pharmaceutically acceptable carrier” refers to a carrier or diluent that does not significantly irritate an organism and does not impair the biological activity and properties of an administered component. The pharmaceutically acceptable carrier used in the present invention may be used by mixing one component or one or more of these components, comprising saline, sterile water, Ringer's solution, buffered saline, a dextrose solution, a maltodextrin solution, glycerol, and ethanol, and if necessary, other conventional additives such as antioxidants, buffers, and bacteriostatic agents may be added, and formulated in the form of an injection suitable for injection into tissues or organs. Also, it may be formulated as an isotonic sterilization solution or, in some cases, a dry preparation (especially a freeze-dried preparation) that can be an injectable solution by adding sterile water or physiological saline. Also, a target organ-specific antibody or other ligands may be attached to the carrier so that the carrier can specifically act on the target organ.

In addition, the composition of the present invention may further comprise a filler, an excipient, a disintegrant, a binder, or a lubricant. Also, the composition of the present invention may be formulated using methods known in the art to provide rapid, sustained, or delayed release of the active ingredient after administration to a mammal.

In one embodiment, the pharmaceutical composition may be an injectable formulation and may be administered intravenously, but is not limited thereto.

In the present invention, the pharmaceutical composition may be accompanied by instructions in connection with the packaging in a form directed by the government agency in charge of the manufacture, use, and sale of drugs, if necessary. The instruction indicates the approval of the agency with respect to the form of the composition or administration to humans or animals, and maybe, for example, a label approved by the US Food and Drug Administration (FDA) for the prescription of a drug.

Meanwhile, in the present invention, “dual-target” means that the dual-targeting lipid-polymer hybrid nanoparticles (T-hNPs) of the present invention target two types of cells. The two types of cells specifically refer to cancer cells and tumor-associated environmental cells. For example, the cancer cells refer to acute myeloid blood cancer cells, and the tumor environment cells refer to leukemia-associated myeloid cells.

In the present invention, “targeting moiety” refers to a substance having a portion to which a receptor can bind in receptor-ligand binding as in antibody-antigen binding. The targeting moiety binds to a target cell in a form in which the receptor is bound at one end, and specifically, the targeting moiety comprises an antibody and a humanized, chimeric, or single-chain form thereof, and other binding fragments and variants thereof.

The binding fragment may comprise, for example, Fab, Fab′, F(ab′)2, Fv, and scFv fragments, but is not limited thereto.

In one embodiment, the targeting moiety may be, for example, a single-chain antibody, and may bind to target cells in a form in which the receptor is bound to the single-chain antibody, that is, in the form of a scFv-monomeric avidin fusion (sFVA) protein.

In addition, the present invention provides a method of preventing or treating cancer comprising administering a therapeutically effective amount of the dual-targeting lipid-polymer hybrid nanoparticles (T-hNPs) comprising the polymer core comprising the heme oxygenase 1 inhibitor and the lipid membrane (shell) comprising the targeting moiety or a composition comprising the same to an individual in need thereof.

In one embodiment, it was confirmed that when the dual-targeting lipid-polymer hybrid nanoparticle (T-hNPs) targets cancer cells, the heme oxygenase 1 inhibitor acts to increase the reactivity of cancer cells to anti-cancer drugs (active targeting), and when targeting tumor-environment cells, the heme oxygenase 1 inhibitor acts to inhibit heme oxygenase 1, which acts as an anti-cancer immune checkpoint agent in macrophages within the tumor environment (passive targeting) (FIG. 1).

Therefore, in the method of preventing or treating cancer, the pharmaceutical composition may be administered in combination with an anticancer agent.

In the present invention, the anti-cancer agent may be selected from the group consisting of dactinomycin, doxorubicin, daunorubicin, ditomycin, and bleomycin, but is not limited thereto.

In the present invention, the cancer may comprise acute myeloid leukemia, bladder cancer, ovarian cancer, breast cancer, prostate cancer, melanoma, metastatic melanoma, lung cancer, non-small cell lung cancer, non-Hodgkin's lymphoma, hepatocellular carcinoma, brain cancer, glioma, or glioblastoma, but is not limited thereto.

The individual used herein refers to a mammal that is the subject of treatment, observation, or experimentation, and preferably refers to a human

In the present invention, the term “administration” means introducing the composition of the present invention to a patient by any suitable method, and the route of administration of the composition of the present invention may be through various routes, either oral or parenteral as long as it can reach the target tissue. The administration may be intraperitoneal administration, intravenous administration, intramuscular administration, subcutaneous administration, intradermal administration, oral administration, topical administration, intranasal administration, intrapulmonary administration, or rectal administration, but is not limited thereto.

In the present invention, the term “effective amount” means an amount necessary to delay the onset or progression of a specific disease to be treated or to entirely alleviate it. In the present invention, the composition may be administered in a pharmaceutically effective amount. It is obvious to those skilled in the art that the appropriate total daily use amount of the pharmaceutical composition can be determined by the treating physician within the range of correct medical judgment.

For the purposes of the present invention, it is preferable to differently apply a specific therapeutically effective amount for a specific patient depending on various factors and similar factors well known in the medical field comprising, but not limited to the type and degree of reaction to be achieved, whether other agents are used, specific composition, a patient's age, weight, general health status, sex and diet, administration time, route of administration, rate of secretion of the composition, duration of treatment, drugs used with or concurrently with the specific composition.

In the present invention, the term “treatment” means an approach to obtain beneficial or desirable clinical outcomes. For the purposes of the present invention, the beneficial or desirable clinical outcomes comprise, but are not limited to, alleviation of symptoms, reduction of disease range, stabilization of disease state (i.e., no exacerbation), delay or decrease in the rate of disease progression, amelioration or temporal alleviation and alleviation of the disease state (partially or entirely), whether detectable or undetectable. “Treatment” may also mean increasing the survival rate compared to the expected survival rate when not receiving the treatment. “Treatment” refers to both therapeutic treatment and prophylactic or preventative measures. The treatments comprise not only the disorder to be prevented, but also the treatment required for a disorder that has already occurred. “Relieving” the disease means that the extent of the disease state and/or undesirable clinical signs are reduced and/or the time course of progression is delayed or prolonged compared to without the treatment.

### Advantageous Effects

The present invention can provide dual-targeting lipid-polymer hybrid nanoparticles (T-hNPs) comprising a polymer core comprising a heme oxygenase 1 inhibitor and a lipid membrane (shell) comprising a targeting moiety, a method of preparing the dual-targeting lipid-polymer hybrid nanoparticles (T-hNPs), a kit for preparing the dual-targeting lipid-polymer hybrid nanoparticles (T-hNPs), a pharmaceutical composition comprising the dual-targeting lipid-polymer hybrid nanoparticles (T-hNPs) as an active ingredient, and a method of preventing or treating cancer comprising administering the pharmaceutical composition to a subject in need thereof.

### DESCRIPTION OF DRAWINGS

FIG. 1 shows the schematic chemo-sensitization in which the dual-targeting lipid-polymer hybrid nanoparticles (T-hNPs) according to the present invention target acute myeloid leukemia (AML) cells, and the heme oxygenase 1 inhibitor acts to increase the reactivity of cancer cells to anti-cancer drugs (active targeting), and the schematic immuno-reprogramming in which T-hNPs target leukemia-associated myeloid (LAM) cells, and the heme oxygenase 1 inhibitor inhibits heme oxygenase 1, which acts as an anti-cancer immune checkpoint in macrophages in the tumor environment (passive targeting).

FIG. 2 shows a preparation and characterization of the scFv-monomeric avidin recombinantly fusion protein of the present invention: a) Structure of the recombinantly fusion protein sFVA. b) SDS-PAGE data of purified sFVA fusion protein. c) His-tag immunodetection of the sFVA. d) A competition assay with anti-CD64 monoclonal antibody. e) A competition assay with avidin-FITC for biotin binding. f) Concentration-dependent cell binding of sFVA/biotin-FITC.

FIG. 3 shows a optimization and characterization of lipid-polymer hybrid nanoparticle of the present invention: a) Optimization of the lipid to PLGA ratio for hybrid nanoparticle preparation. b) Optimization of heme oxygenase 1 (HO 1) inhibitor to polymer (e.g., PLGA) ratio for hybrid nanoparticle preparation. c) Scanning electron microscopy image of empty- and HO1-loaded-hybrid nanoparticles.

FIG. 4 shows a optimization and characterization of lipid-polymer hybrid nanoparticle of the present invention: a) Stability test of the hybrid nanoparticle. b) Optimization of sFVA-bind hybrid nanoparticles. c) Drug loading efficiency and encapsulation efficiency test. d) Drug release test.

FIG. 5 shows a enhanced cellular uptake of hybrid nanoparticle in leukemia cells according to the present invention: In vitro cellular uptake of Cy5-loaded hybrid nanoparticle in leukemia cells.

FIG. 6 shows a enhanced cellular uptake of hybrid nanoparticle in leukemia cells according to the present invention: Confocal microscopy image of cellular uptake of hybrid nanoparticles (Cy5, Red) in the cells at 1 hr after treatment of nanoparticles at a specific concentration. Cells were stained with anti-CD33 antibody (green) for morphology imaging Scale bar: 20 μm.

FIG. 7 shows a biodistribution of lipid-polymer hybrid nanoparticle in sFVA-mediated bone marrow leukemia cell targeting and acute myeloid leukaemia model according to the present invention: a) Experimental procedures and FACS gating strategy for bone marrow cells. b) Representative dot plot of dual targeting lipid-polymer hybrid nanoparticles uptake for human CD33+bone marrow U937 cells. c) Representative dot plot of dual targeting lipid-polymer hybrid nanoparticles uptake for mouse CD45+CD11b+bone marrow immune cells. d) Bar graph for the percentage of lipid-polymer hybrid nanoparticles uptake for U937 and mouse immune cells.

FIG. 8 shows a biodistribution of lipid-polymer hybrid nanoparticle in sFVA-mediated bone marrow leukemia cell targeting and acute myeloid leukaemia model according to the present invention: a) Representative organ image for biodistribution of hybrid nanoparticles (lipid-polymer hybrid nanoparticles and/or dual targeting lipid-polymer hybrid nanoparticles) according to the present invention. b) Total radiant efficiency for the organ distribution of hybrid nanoparticles at 24 hours after injection of hybrid nanoparticles according to the present invention. c) Average radiant efficiency for hybrid nanoparticles in femur and tibia.

FIG. 9 shows a in vitro chemo-sensitization effects of hybrid nanoparticles according to the present invention in leukemia cells: a) A Western blot image of DNR-responsive HO1 overexpression in leukemia cells. b) A Cell viability test in THP-1 leukemia cells. c) A Cell viability test in U937 leukemia cells.

FIG. 10 shows a in vitro chemo-sensitization effects of hybrid nanoparticles according to the present invention in leukemia cells: Apoptosis assay for chemo-sensitization by dual targeting lipid-polymer hybrid nanoparticles according to the present invention in leukemia cells.

FIG. 11 shows a in vitro chemo-sensitization effects of hybrid nanoparticles according to the present invention in leukemia cells: A Bar graph of apoptosis assay in a FIG. 10.

FIG. 12 shows a combination therapy of the dual lipid-polymer hybrid nanoparticles with anti-cancer agents (e.g., daunorubicin) suppresses the leukemia growth: A experimental schedule for an in vivo therapeutic study.

FIG. 13 shows a combination therapy of the dual lipid-polymer hybrid nanoparticles with anti-cancer agents (e.g., daunorubicin) suppresses the leukemia growth: a) A leukemia growth in bone marrow. b) A leukemia growth in liver. c) A representative spleen images.

FIG. 14 shows a combination therapy of the dual lipid-polymer hybrid nanoparticles with anti-cancer agents (e.g., daunorubicin) suppresses the leukemia growth: a) Bar graph for spleen weight. bl) Apoptosis assay for human CD33+U937 cells in bone marrow. b2) bar graph indicates live cells (Annexin V−, 7AAD−) and apoptotic cells (Annexin V+). c) qRT-PCR analysis.

FIG. 15 shows a immune reprogramming and activation effect of dual targeting lipid-polymer hybrid nanoparticle according to the present invention in bone marrow myeloid cells: a) Gating strategy for bone marrow myeloid cells. b) The ratio of CD11b+myeloid cells to CD45+ total immune cells. c) The ratio of F4/80-hi, CD206-M1-like macrophages in total myeloid cells. d) The ratio of Gr1-int and F4/80-int monocytic cells in total myeloid cells. e) The ratio of Ly6c-int and Ly6c-himonocyte in total bone marrow myeloid cells.

FIG. 16 shows a immune reprogramming and activation effect of dual targeting lipid-polymer hybrid nanoparticle according to the present invention in bone marrow myeloid cells: a) A flow cytometric analysis of intracellular IL-12p70 expression in bone marrow CD11b+Ly6c+monocytes. b) A flow cytometric analysis of intracellular TNF-α expression in bone marrow CD11b+Ly6c+monocytes. c) Immune activation and monocyte/macrophage activation marker gene expression levels in bone marrow. d) Immune suppression and M2-like macrophage marker gene expression levels in bone marrow.

FIG. 17 shows a immune reprogramming and activation effect of dual targeting lipid-polymer hybrid nanoparticle according to the present invention in bone marrow myeloid cells: A magnetic cell sorting for CD11b+myeloid cells and experimental scheme for ex vivo analysis. Bone marrow cells were harvested from C57BL/6 mice and analyzed by flow cytometry before and after sorting.

FIG. 18 shows a immune reprogramming and activation effect of dual targeting lipid-polymer hybrid nanoparticle according to the present invention in bone marrow myeloid cells: a) Marker gene expression levels in HO1-inhibited CD11b+myeloid cells in response to apoptotic leukemia. b) Marker gene expression levels in HO1-inhibited CD11b+myeloid cells in response to chemotherapy.

FIG. 19 shows a survival study and therapeutic mechanisms of chemo- and immuno-combination therapy by dual targeting lipid-polymer hybrid nanoparticles according to the present invention: a) A experimental schedule for survival study. b) Survival study (n=4-5 per group). c) Body weight graph.

FIG. 20 shows a survival study and therapeutic mechanisms of chemo- and immuno-combination therapy by dual targeting lipid-polymer hybrid nanoparticles according to the present invention: A experimental summary of therapeutic mechanism for dual targeting lipid-polymer hybrid nanoparticles according to the present invention.

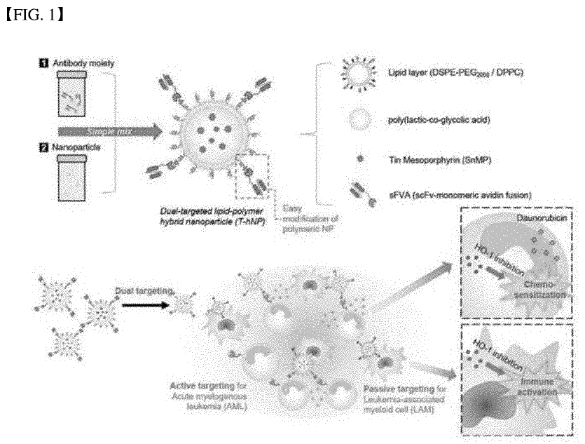

### MODES OF THE INVENTION

Advantages and features of the present invention and a method of achieving them will become apparent with reference to the embodiments described below in detail. However, the present invention is not limited to the embodiments disclosed below and may be implemented in a variety of different forms. Only the present embodiments are intended to complete the disclosure of the present invention and are provided to completely inform the scope of the invention to those of ordinary skill in the technical field to which the present invention belongs, and the invention is only defined by the scope of the claims.

### EXAMPLES

Materials

SnMP, DNR hydrochloride, poly(lactic-co-glycolic acid) (PLGA, lactide: glycolide 50:50, 7000-17000 Da), and biotin-FITC were purchased from Sigma Aldrich (St. Louis, Mo., USA). 1,2-Dipalmitoylsn-glycero-3-phosphocholine (DPPC) and 1,2-distearoyl-sn-glycero-3-phosphoethanolamine-N-[biotinyl(polyethylene glycol)-2000] (DSPEPEG2000-Biotin), 1,2-distearoyl-sn-glycero-3-phosphoethanolamine-N-[amino(polyethylene glycol)-2000] (DSPE-PEG2000) were obtained from Avanti Polar Lipids, Inc. (Alabaster, Ala., USA). Anti-human CD33, CD64 antibodies and anti-mouse CD11b, CD45, CD206, Ly6c,Gr1, TNF-α IL12p70, Rat IgG1 Isotype antibodies were purchased from BD Biosciences (USA).

Anti-mouse F4/80 antibody and Avidin-FITC were purchased from Biolegend (San Diego, Calif., USA). Antihuman CCR2 antibody was obtained from R&D Systems (Minneapolis, Minn., USA). Anti-His-Tag, anti-human HO1 (P249), and β-actin antibodies (13E5) were obtained from Cell Signaling Technology (Danvers Mass., USA).

Vector Construction

A 429 and 888 base pair sequence formonomeric avidin and anti-CD64 scFv were cloned (Incorporation Bioneer, Korea) in pET21a (Novagen, Madison, Wis.) by NotI, xhoI and xbaI, NotI sites, respectively, for bacterial expression.

Hybrid Nanoparticle Preparation

DSPE-PEG2000 (ratio of 5:1 for biotinylated to non-biotinylated) andDPPC were mixed at a molar ratio of 1:3 and stored for 1 h at room temperature to evaporate the chloroform. The prepared lipid mixture was hydrated in water (4% EtOH, 10 mL) at 0.2 mg mL⁻¹ and gently stirred. SnMP (400 μg) and PLGA (7.2 mg) solutions were prepared at concentrations of 4 mg mL⁻¹ in dimethyl sulfoxide (DMSO) and 2.4 mg mL⁻¹ in dichloromethane, respectively. The drug/PLGA solution (836 μL) was dropped slowly to a lipid solution (2.4 mL) at a ratio of 1:3 (v/v, PLGA: lipid), sonicated and evaporated to remove the dichloromethane. The prepared particle solution (1 mg mL⁻¹) was concentrated and washed through a cellulose membrane (MWCO 30 000 Da) at 2.5⁻¹⁰ mg mL⁻¹.

sFVA Protein Expression and Purification

BL21 (DE3) cells (Novagen, Madison, Wis.) were transformed with a sFVA-cloned pET21 a vector and cultured in 20 mL of Amp+ lysogeny broth (LB) at 37° C. After 2-4 h of incubation, the cells were cultured in 0.5 L of LB medium. When the optical density at 600 nm reached 0.2-0.3, 1 mm isopropyl β-D-1-thiogalactopyranoside (IPTG) was added and the cells were induced for 4 h at 37° C. The induced pellet was re-suspended in a lysis buffer (pH 8.0) and then sonicated (pulse on: 20 s, total 2 min, off: 59 s, amplitude: 30%). The protein solution was then collected through centrifugation at 27 500 g, and the resulting solution was filtered using a 0.45 μm filter.

Affinity Chromatography Purification

The protein solution was loaded to a Ni-NTA agarose resin (Qiagen)-charged column and washed with 40 volume equivalents of washing buffer. The resin-bound protein was eluted at 250 mm imidazole elution buffer. The purified protein was dialyzed using a Slide-A-Lyzer Dialysis cassettes (Thermo Fisher Scientific, 12 mL, CA; MWCO 10 000 Da) in presence of a refolding buffer (pH 8.2) and dialyzed through a phosphate buffered saline (PBS) at pH of 7.4. Protein was concentrated through a cellulose membrane (MWCO 10 000 Da).

Cell Culture

Human THP-1 and U937 leukemia cells were purchased from ATCC (Virginia, USA) and cultured at 37° C. in 5% CO2 in RPMI1640 medium (Welgene, Korea) supplemented with 10% fetal bovine serum and 1% penicillin. Cells were passaged to a density of 1-2 ×10⁵ cells mL⁻¹ and media was changed every 2-3 days.

SDS-PAGE and Western Blotting

Purified and PBS-dialyzed protein was mixed with Laemmli buffer (5 mm dithiothreitol), boiled for 15 min and loaded into 12% SDS-PAGE gels for electrophoresis. Gel was stained with Coomassie blue or transferred to a polyvinylidene fluoride membrane (Millipore, Billerica, Mass.) for immunodetection by anti-His-Tag antibody (Cell Signaling Technology, Danvers Mass., USA) and anti-rabbit IgG antibody-HRP (Santa Cruz, Tx., DA, USA).

Competitive Binding Study

THP-1 cells (2×10⁵ cells well⁻¹) were incubated with anti-CD64 mAb-FITC (BD Pharmingen) in the presence of sFVA (10 μg mL⁻¹) in PBS at 4° C. for 20 min. After the cells were washed twice, they were analyzed using FACSCalibur (BD Biosciences, USA). For biotin-competitive binding of sFVA, the THP-1 cells were incubated with anti-CCR2 mAb-biotin in the presence of sFVA and avidin-FITC.

Drug Loading Efficiency and Release Profiling

After preparation of SnMP-loaded hNPs (10 mg mL⁻¹), 0.6-0.7 mg particles were used to measure the loading efficiency and encapsulation efficiency at absorbance 399 nm using Tecan. The 10 mg mL⁻¹ particle was resuspended in PBS (DMSO 10%) and centrifuged to harvest the released medium at 6, 12, 24, 48, and 72 h. Release media and particle were freeze-dried and resuspended for detection of SnMP at absorbance 399 nm.

Characterization ofHybrid Nanoparticles

The prepared particles (1 mg mL⁻¹) were diluted in water and analyzed with Zeta-Sizer (Malvern) to optimize lipid/PLGA, particle/drug, and particle/sFVA ratios. The size of the hybrid particles (10 mg mL⁻¹) was measured at indicated days and weeks after preparation to evaluate stability.

Cellular Uptake and Confocal Microscopy Imaging

THP-1 and U937 cells (1×10⁶ cells mL⁻¹) were incubated with Cy5-loaded PLGA and hNPs at a concentration of 5 pg mL⁻¹ for 1 h and analyzed by flow cytometry. Cells were stained with DAPI Fluoromount-G (Southern Biotech) and imaged by confocal microscopy (Leica).

Daunorubicin-Responsive HO1 Upregulation in Leukemia Cells

THP-1 and U937 cells (4×10⁵ cells mL⁻¹) were seeded and cultured in complete medium with various concentrations of DNR for 24 h. The cells were lysed using RIPA buffer and total protein was used for immunodetection by using anti-human HO1 and kactin antibodies (Cell Signaling Technology, Danvers, Mass., USA).

Cell Viability Test and Apoptosis Assay

Seeded THP-1 and U937 cells (4-5×10⁵ cells mL⁻¹, 24 well plate) were treated with hNPs (SnMP concentration: 1,3,5 μm). 5 h after treatment, DNR was added and incubated for an additional 24 and 30 h for further analysis. Total cell numbers were calculated using a hemocytometer. For apoptosis assay, cells were stained with Annexin V and 7AAD (BD Biosciences, USA) and analyzed by flow cytometry.

Orthotopic Acute Myelogenous Leukemia Modeling

4-6 week-old male NOD-SCID il2r gamma −/− (NSG) mice (Jackson Laboratory) were intravenously injected with 1-2×10⁶ U937 cells and their survival was evaluated under SPF conditions. All animal experimental procedures were reviewed and approved by the Institutional Animal Care and Use Committee (IACUC) of Hanyang University (2019-0076A) and were performed in accordance with the relevant guidelines.

In Vivo Leukemia-Targeted Delivery of Hybrid Nanoparticle

1 week post cell infusion, U937-bearing NSG mice were intravenously injected with hNP and T-hNP (Cy5, 0.6 mg kg⁻¹) and after 2 h, bone marrow cells were harvested from the femur and tibia and filtered through a 100 μm filter. Red blood cells were lysed and stained with anti-human CD33, mouse CD11b, and CD45 antibodies for flow cytometric analysis.

In Vivo Biodistribution of Hybrid Nanoparticle

At day 10 post U937 cell infusion of NOD-SCID il2r gamma −/− mice, hNPs were intravenously injected (Cy5, 0.6 mg kg⁻¹). The mice were sacrificed and Cy5 fluorescence intensity was measured in major organs at 4 and 24 h post injection using VISQUE InVivo Smart (Vieworks Co, Korea) in the Korea Basic Science Institute (Chuncheon, Korea).

In Vivo Therapeutic Study in an Orthotopic Model

U937-bearing NSG mice were intravenously injected with hNPs (SnMP dose: 1.4 mg kg⁻¹) at 4, 6, 8, 10 days post cell infusion. At day 11, major leukemia niche organs were harvested and analyzed for further experimental analysis. Bone Marrow Myeloid Cell Analysis and Gene Expression: After treatment, total bone marrow cells were harvested from femur and tibia and filtered through a 100 μm filter. Red blood cells were lysed and stained with myeloid cell lineage markers for flow cytometry analysis. Total RNA was isolated from bone marrow cells and reverse transcribed to cDNA using iScript cDNA synthesis kit (Bio-Rad) to measure marker gene expression levels. All primers were synthesized and purchased from IDT DNA.

Ex Vivo Myeloid Cell Reprogramming

Total bonemarrow cells were isolated from 5-7 week-old C57BL/6mice (Orient Bio) and sorted using magnetic EasySep Mouse CD11b positive Selection Kit (STEMCELL Technologies, USA). Purity was validated by FACSCalibur (BD Biosciences, USA) and seeded (4×10⁵ cells mL ⁻¹). 24 h post treatment of hNP, DNR, and DNR-exposed U937 cells (DNR: 0.2 μm) were added. Total RNA was isolated, and reverse transcribed to cDNA (iScript cDNA synthesis kit, Bio-Rad) and gene expressions weremeasured. For DNR-exposed U937, U937 cells were exposed to DNR for 5 h and washed twice and added to myeloid cells (8×10⁴ cells mL⁻¹).

In Vivo Therapeutic and Survival Study

4-6 week-old NSG mice were injected with 1×10⁶ U937 cells intravenously via tail vein injection. At days 1, 3, 5 7, 9, and 11 post cell infusion, hNPs (SnMP: 1.4 mg kg⁻¹) and DNR (1.5 mg kg⁻¹) were intravenously injected and further monitored for survival rate.

Statistical Analysis

All data are presented as mean ±SD and SEM. Statistical analyses were performed using a Student's t-test and one-way ANOVA with Tukey's post-hoc test in GraphPad Prism 7 Project software. All animal studies were analyzed using a non-parametric Kruskal-Wallis test.

Results

1. Preparation and Characterization of Engineered Antibody Fusion Protein, sFVA

The anti-CD64 scFv was recombinantly fused to monomeric avidin (sFVA) for nanoparticle modification.

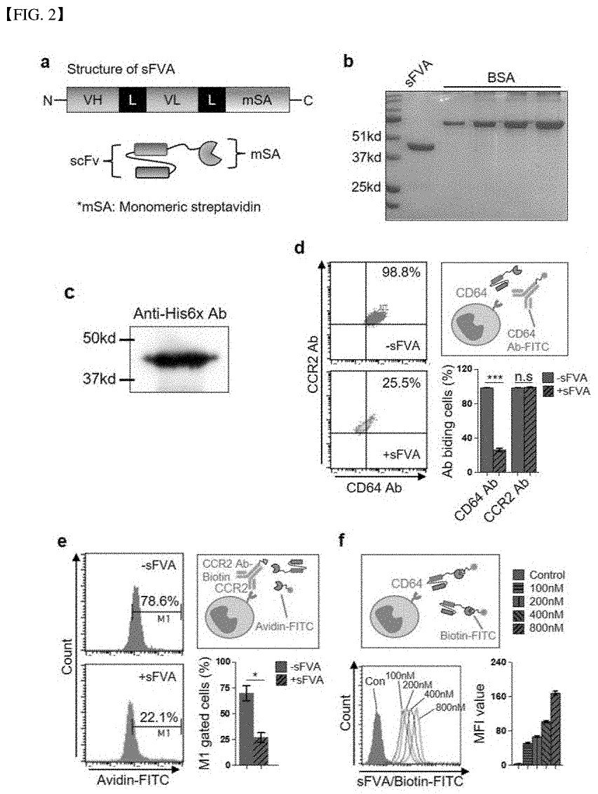

The sFVA was expressed and purified from a bacterial expression system and dialyzed to recover its antigenbinding ability. As shown in FIG. 2b and FIG. 2c, the sFVA band was identified at the molecular weight of 43 kDa, which is consistent with its theoretically estimated molecular weight. To evaluate human CD64- and biotin-binding abilities, sFVA was competed with commercial antibodies.

The CD64-expressing THP-1 cells showed reduced anti-CD64 antibody-FITC binding in the presence of sFVA with 3.75-fold lower % of cell binding compared to the non-competed group, while anti-CCR2 antibody-PE did not compete with sFVA (FIG. 2d).

A competition assay with anti-CCR2 mAb-biotin and avidin-FITC proved the biotin-binding affinity of sFVA with 2.6-fold lower % of cell binding in the presence of sFVA compared to the non-competed group (FIG. 2e). Biotin-FITC-bound sFVA exhibited concentration-dependent cell binding profiles (FIG. 2f).

2. Optimization and Characterization of PLGA-Lipid Hybrid Nanoparticles Lipid-layered polymeric hNPs have been reported as efficient drug delivery carriers for cancer cells and T cells.

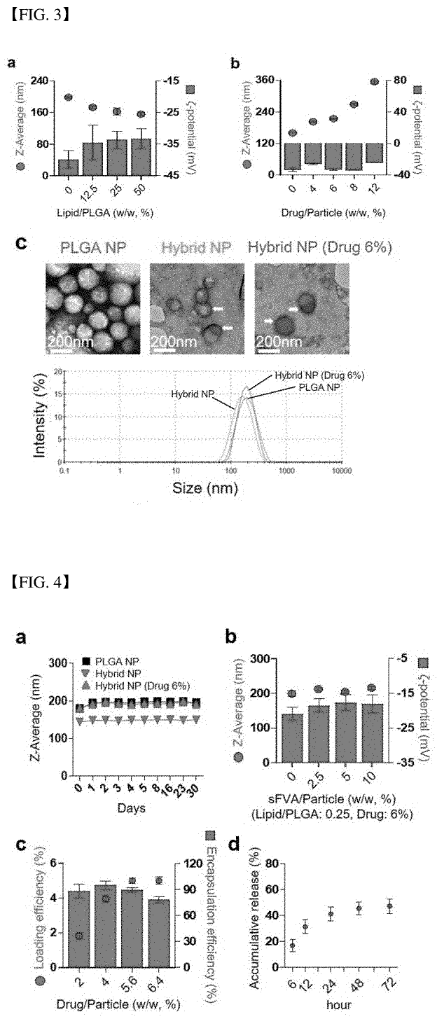

To develop an HO1-inhibitor-loaded hNP, a PLGA-polymeric core was complexed with various ratios of DSPE-PEG2000 and DPPC (at a molar ratio of 1:3) as previously described. The lipid weight ratio to PLGA of 0.25 indicated an increased (ζpotential with 33.7±2.71 mV and an average size of 162.9±8.64 nm in comparison with −39.86±2.85 mV and 198.5±2.06 nm of PLGA nanoparticles (FIG. 3a). SnMP is an FDA-approved HO1 inhibitor and has been used to treat hyper bilirubinemia. Among various SnMP to particle ratios, 4-6% were found to be the optimum loading amount without affecting the size and potential of the prepared nanoparticles with 214.5±0.7 nm in 6% drug amount loading (FIG. 3b). As shown in FIG. 3c, scanning electron microscopy imaging of prepared hNPs revealed that the PLGA particle is layered by a thin lipid membrane (indicated as an arrow).

After preparation and concentration, the hNP retains its spherical shape, size, and poly dispersity index of 0.1-0.2 formore than a month. The SnMP-loaded hNP is slightly larger than an empty hNP with 181±3 and 144.1±2.4 nm, respectively (FIG. 4a).

Finally, sFVA was complexed with hNP for binding on DSPEPEG2000-biotin, with a weight to hNP ratio of 2.5-5% indicated as an optimal formulation (FIG. 4b). In FIG. 4c, the drug loading efficiency was 4.99±0.15% and 4.98±0.21% for 5.6% and 6.4% of initial drug loading, respectively, and which is converges to weight ratio 5% of hNP. The drug release study in FIG. 4d shows 47.08±5.45% of the drug was released from the hNP after 72 h at 37° C.

3. Enhanced Cellular Uptake of Hybrid Nanoparticle in Leukemia Cells

To evaluate enhanced cellular uptake by lipid-layer and sFVAmodification, THP-1 and U937 cells were incubated with Cy5-loaded nanoparticles and analyzed by flow cytometry.

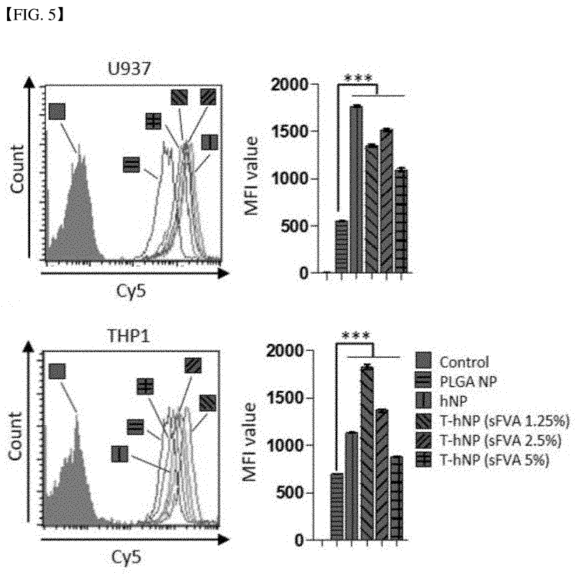

The size and (ζpotential of Cy5-loaded hNP were comparable with SnMP-loaded hNP. In human AML cell lines (CD64+) THP-1 and U937, hNPs showed 1.62-and 3.2-fold higher cellular uptakes in comparison with PLGA nanoparticles. However, sFVA-modification on the surface of hNP at a weight ratio 1.25-5% exhibited different patterns in cellular uptake enhancements between two cell lines. In U937, sFVA-modification reduced cellular uptake of hNP which differed from enhanced cellular uptake by 1.25-2.5% sFVA modification in THP-1 cells, demonstrating different cellular uptake mechanism by lipid-cell membrane interaction between these two cell lines (FIG. 5). Confocal microscopy imaging showed similar Cy5 uptake patterns with flow cytometry data. In comparison with PLGA nanoparticle, hNPs with/without sFVA modification showed higher cellular uptake and were mostly distributed in cytoplasm upon surface binding and internalization (FIG. 6).

Collectively, the hNP and sFVA-modified hNP (T-hNP) exhibited higher cellular uptakes than PLGA nanoparticles. Although higher sFVAmodification hampered cellular internalization of hNP in vitro, targeted deliverywith antibody was expected to represent more prominent effects in vivo. Therefore, 2.5% and 5% sFVA-modification were chosen for in vivo study.

4. sFVA-Mediated Bone Marrow Leukemia Cell Targeting and Biodistribution of Hybrid

Nanoparticle in U937-Bearing Orthotopic AML Model

The CD64+ CD33+ U937 cells were injected intravenously into NSG mice and formation of human xenograft model was validated.

Human U937 cells are commonly accumulated in liver and bone marrow niches followed by enlarged spleens which recapitulate human AML pathologies. Bone marrow is a clinically relevant, dominating organ in blood cancers, and leukemia-targeted delivery was evaluated in bone marrow.

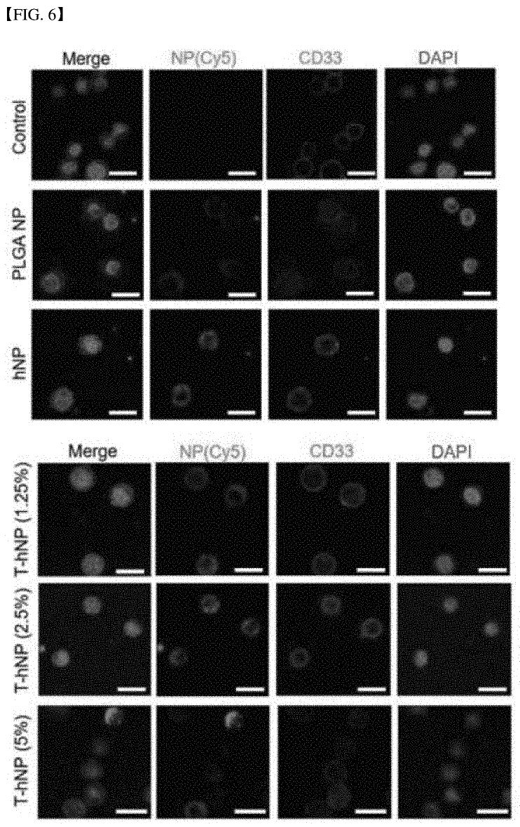

The hNP and sFVA-modified T-hNP were injected into an orthotopic AML model and their uptake into bone marrow leukemia cells was analyzed from the tibia and femur by using flow cytometry (FIG. 7a).

As shown in FIG. 7b, human CD64 + CD33+ U937 cells showed cellular uptake of 79.8±7.2% for T-hNP (5% sFVA) and 35±6.9% for hNP. In addition, sFVA-modification at 5% resulted in higher leukemia cell-targeted uptake than 2.5% In FIG. 7c, hNP was shown to be internalized by 33.5±4.3% of mouse CD11b+ bone marrowmyeloid cells and T-hNP showed a slightly reduced uptake by 27.5±3.3%, which confirmed that sFVA-modification enhanced leukemia cell-targeted uptake of hNP.

It should be pointed out that only 10.1±1.7% of the CD11b-immune cells internalized ThNP (FIG. 7d). Macrophages and monocytes are mononuclearphagocytes naturally engulfing nanoparticles more than other cell types.

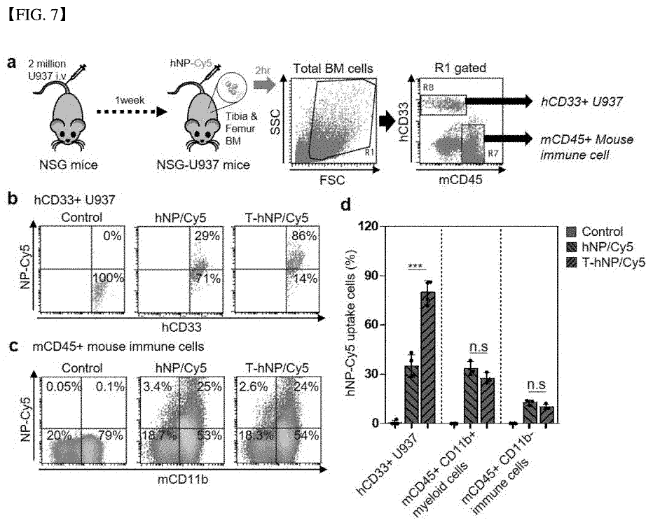

Also, the negatively charged surface of nanoparticles was shown to enhance phagocytic- and myeloidcell uptake. At 10 days post cell infusion, orthotopic AML xenografts were intravenously injected with Cy5-loaded hNP and T-hNP.Major organs and femur and tibia were harvested tomeasure fluorescence intensity. Both hNP and T-hNP highly localized to liver and kidney which are major clearance routes for nanoparticles (FIG. 8a). The hNP and T-hNP localization in femur and tibia was quantified and compared with other organs. In comparison with hNP, T-hNP showed higher accumulation in liver, lung, and femur and tibia, which are attributable to leukemia-enriched organ targeting effects (FIG. 8b).

As above described, liver and bone marrow are major U937 accumulation organ and lung is also a probable organ due to the size of cells. Average radiant efficiency analysis in femur and tibia of T-hNP group showed 1.3-fold higher intensity compared to hNP group which is reasonable to explain bone marrow leukemia-targeted delivery by sFVA-modification (FIG. 8c). And we confirmed that U937 cells comprise 10% to 25% of bone marrow cell at 10 days post AML modeling.

Collectively, sFVA-modification enhanced active targeting of nanoparticles to CD64 + leukemia cells in bone marrow and leukemia niche organs, and passively targeting to CD11b+ myeloid cells.

5. In Vitro Chemo-Sensitization Effect of HO1-Inhibiting Hybrid Nanoparticle in Leukemia

Cells

To evaluate the chemo-sensitization effect of HO1-inhibiting hNPs, THP-1, and U937 cells were treated with emptyand SnMP-loaded T-hNPs in the presence of daunorubicin (DNR), a first-line chemotherapeutic for AML.

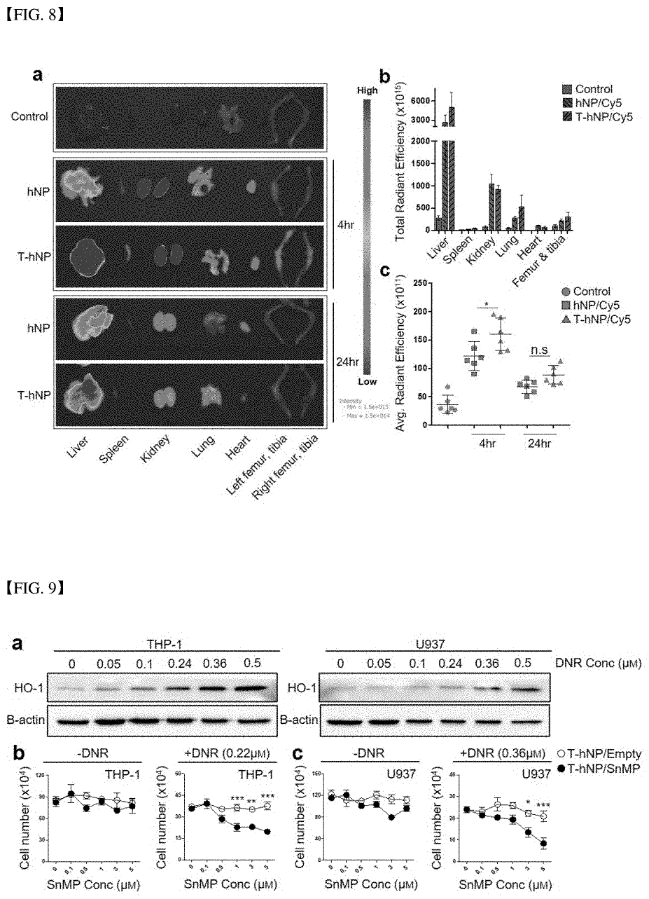

The HO1 was overexpressed depending on the concentration of DNR in the THP-1 and U937 cells (FIG. 9a). In FIG. 9b and FIG. 9c, T-hNP/SnMP improved the cytotoxic effect of DNR at SnMP concentrations of 1 to 5 μm.

However, no cytotoxic effects were observed in the absence of DNR. Flow cytometry data revealed increased apoptotic responses of leukemia cells to DNR at various concentrations of T-hNP/SnMP compared to T-hNP/Empty group (FIGS. 10 and 11).

6. Combination Therapy of HO1-Inhibiting T-hNP with Daunorubicin Suppresses Leukemia Growth in Human AML-Bearing Orthotopic Model

A human U937 AML xenograft model has been used to distinguish mouse myeloid cells from human cells, which facilitated experimental analysis of immune reprogramming in bone marrow niche myeloid cells. Recent study showed that HO1 acted as an immune checkpoint molecule in myeloid cell and a combination therapy of SnMP with 5-FU boosted antitumor immune response in breast tumor model.

In several prior studies, many kinds of chemotherapeutics induce anti-cancer immune responses. Additionally, most of immunotherapeutic reagents show outstanding anti-tumor effect when only it combined with chemotherapeutic and other immunotherapeutic. Based on the chemo-sensitization effect and immune checkpoint function of HO1, T-hNP/SnMP was combined with DNR in human AML-bearing orthotopic model. Empty T-hNP +DNR group represents chemotherapy by DNR and T-hNP/SnMP +DNR group represents chemoand immuno-combination therapy.

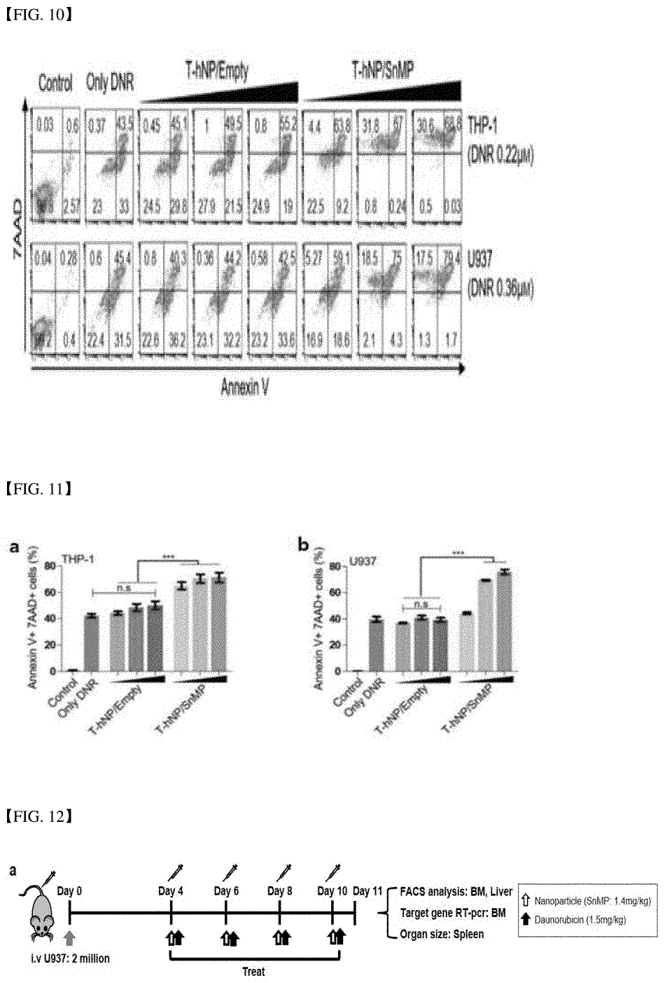

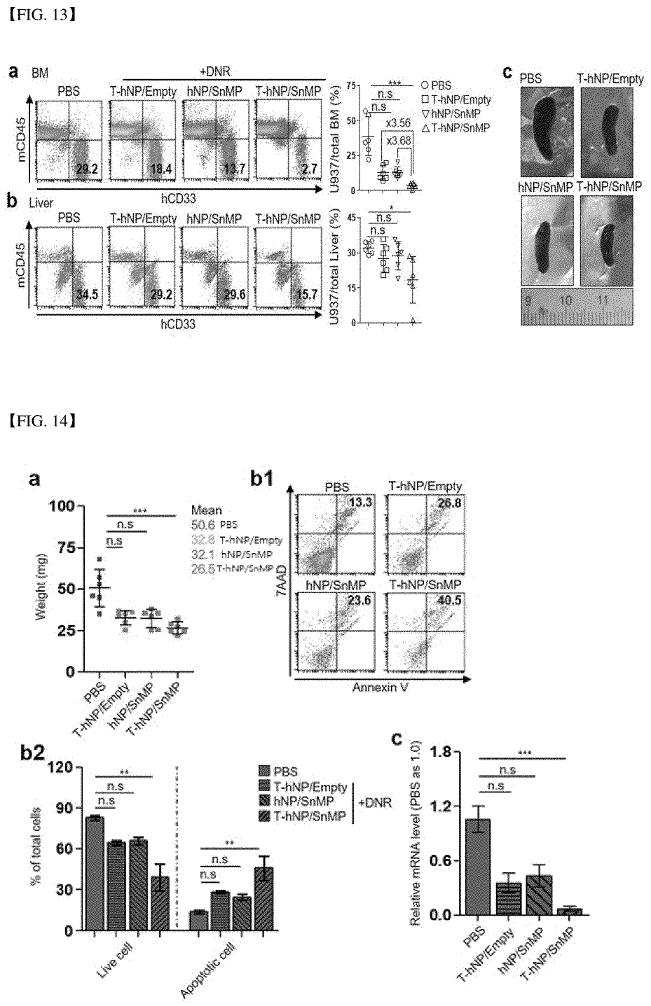

First, the anti-cancer effect of HO1-inhibiting T-hNP was evaluated in an orthotopic AML model. Xenograft mice were injected 4 times with nanoparticles and treated with DNR, and their organs were analyzed at day 11 (FIG. 12). As shown in FIG. 13a, leukemia cell growth in bone marrow was significantly reduced in the T-hNP/SnMP +DNR treatment group, at a rate that was 3.68-fold and 3.56-fold lower than with hNP/SnMP +DNR and T-hNP/Empty +DNR groups, respectively. The T-hNP/SnMP +DNR also suppressed growth of liver-enriched leukemia cells (FIG. 13b). In FIG. 13 c and FIG. 14a, the ThNP/SnMP +DNR group showed highly reduced splenomegaly. Furthermore, 1.63-fold and 1.87-fold more apoptotic CD33+U937 cell populations were detected in the bone marrow of the T-hNP/SnMP +DNR treatment group in comparison with ThNP/Empty and hNP/SnMP groups, respectively (FIG. 14 1b and FIG. 14 b2).

Less amount of human GAPDHmRNA was measured in the bone marrow of the treatment group, which is consistent with the flow cytometry results of FIG. 13a.

7 Immune Reprogramming and Activation Effect of HO1-Inhibiting T-hNP in Bone Marrow Myeloid Cells

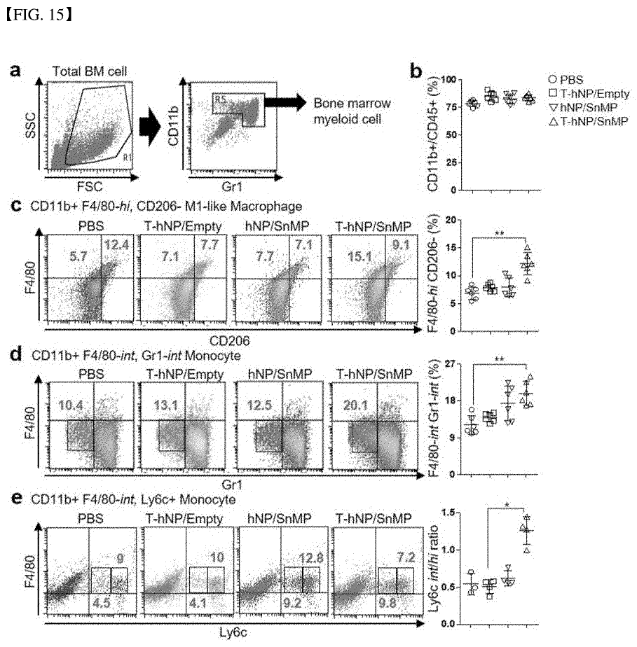

To validate the immune reprogramming and activation effects of HO1-inhibiting T-hNP, mouse bone marrow myeloid cells were analyzed by flow cytometry. As shown in FIG. 15a, CD11b+ cells were gated as total bone marrow myeloid lineages. The total CD11b+ myeloid cell % to CD45+ immune cell did not change significantly between groups (FIG. 15b). The F4/80-hi CD206-M1-like and F4/80-hi CD206+M2-like macrophages were analyzed, and CD206-M1-like cells were increased in ThNP/SnMP group with 12.1±2% compared to 7.83±0.66% and 7.9 ±1.7% in T-hNP/Empty and hNP/SnMP, respectively (FIG. 15c).

However, F4/80-hi CD206+ M2-like macrophage was not significantly reduced. The M1/M2 ratio of T-hNP/SnMP +DNR group was also higher than other groups. Gr1-intermediate (Gr1-int) and F4/80-intermediate (F4/80-int) myeloid cells were increased in the hNP/SnMP +DNR and ThNP/SnMP +DNR groups with 17.3±4.2% and 19.8±2.9%, respectively. Gr1 is Ly6c/Ly6G and Gr1-int, F4/80-int cells are generally monocytic lineages, and CJ Perry et al. demonstrated Chi313+Ly6c+ F4/80-intmonocyte attraction in melanomas after myeloid-targeted immunotherapy, which is a polyfunctional inflammatory cell with increased cytokine expression.

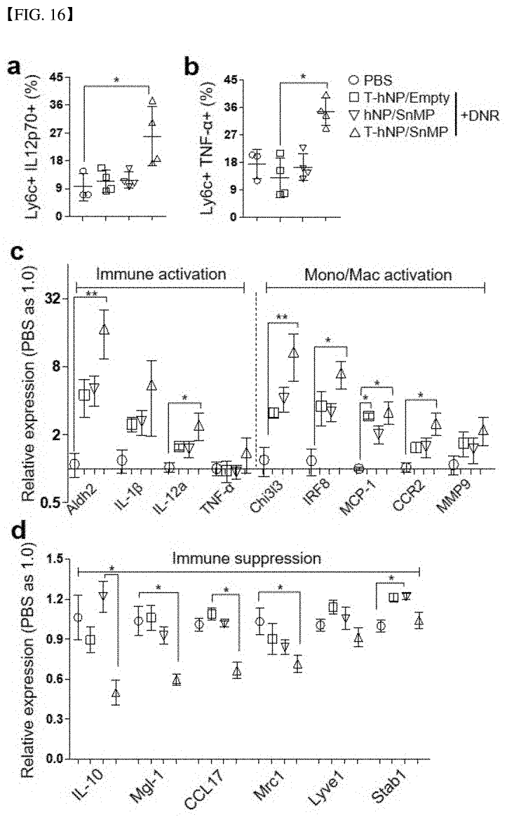

Total Ly6c+ monocytic cell % was not significantly different between groups. However, the ratio of Ly6c-int to Ly6c-hi monocytes was increased in T-hNP/SnMP +DNR group of 1.26±0.1 in comparison with 0.5±0.09 and 0.6±0.1 of T-hNP/Empty+DNR and hNP/SnMP +DNR, respectively (FIG. 15e). These results demonstrate Ly6c-int monocyte recruitment and phenotypic change of monocyte population. Furthermore, in FIG. 16a and FIG. 16b, 2.2-fold and 2.3-fold higher % of CD11b+ Ly6c+ monocytes from T-hNP/SnMP +DNR expressed intracellular levels of inflammatory cytokines, IL12p70 and TNF-α′, respectively. Nonmonocytic Ly6c-CD11b+ cells show less prominent upregulation of intracellular cytokine Immune activation, suppression, and monocyte/macrophage gene expressions were analyzed in bone marrow cells to evaluate the immune reprogramming effect of T-hNP/SnMP treatment Immune activation-relevant genes such as IL-12a, IL-1,g, and Aldh2 were mostly upregulated in ThNP/SnMP+DNR in comparison with other groups, and monocyte/macrophage activation markers such as interferon regulatory factor 8 (IRF8) and CCR2 were increased, which was consistent with the result of monocyte/macrophage phenotype change in flow cytometric analysis. In previous studies, IRF8 activation demonstrated immunotherapeutic effect and human leukemia inhibition. The increased level of Chi313 was reasonable to explain by Chi313+Ly6c+ polyfunctional monocyte attraction. M2-like macrophage and immune suppression-relevant gene expressions showed decreased levels of IL-10, Mgl-1, and Mrcl (CD206) in the T-hNP/SnMP +DNR treatment group (FIG. 16d).

IL-10 is a major immune-suppressive cytokine and Mgl-1 is a C-type lectin receptor for glycan and related with tumorassociated macrophage and immune suppression. A reduced chemokine, CCL17 was associated with unfavorable prognoses of tumors and attraction of regulatory T cells in a previous study. In comparison with T-hNP/SnMP, only a modest change in gene expression was measured in the hNP/SnMP group even comparable Gr1-int, F4/80-int monocytic cell recruitment. As an immune checkpoint molecule in myeloid cells, HO1-inhibition shows therapeutic effect only when it combined with chemotherapeutics suggesting that chemo-induced specific conditions trigger HO1-inhibition-mediated immune activation.

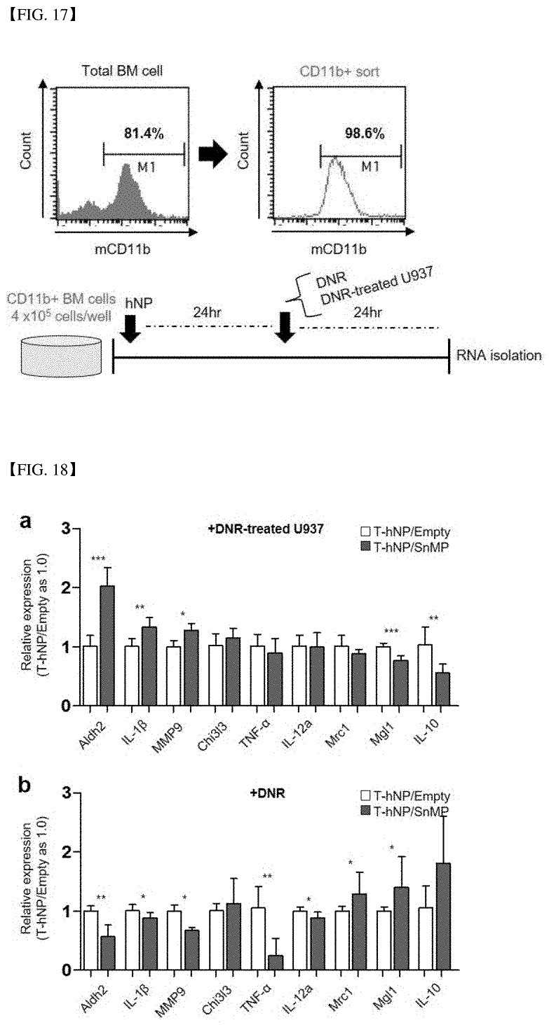

To understand the improved anti-leukemic and immune activation mechanism of the T-hNP/SnMP group, CD11b+ bone marrow cells were sorted by magnetic beads and analyzed ex vivo (FIG. 17). The magnetic sorted CD11b+ cell showed ≈98.6% purity and exposed to DNR or DNR-treated U937 at 24 h after T-hNP treatment, could discriminate immune activation response to DNR from response to apoptotic leukemia cells (DNR-treated U937). As shown in FIG. 18a and FIG. 18b, T-hNP/SnMP treatment modulates expression levels of listed-genes as comparable to in vivo analysis. The T-hNP/SnMP treated myeloid cells responded strongly to apoptotic leukemia cells with increased inflammatory genes and reduced immune suppressive gene expression. Interestingly, HO1-inhibition-mediated gene expression was reversed in response to DNR treatment.

Collectively, HO1-inhibiting T-hNP reprogrammed bone marrow myeloid cells by recruiting Gr1-int, Ly6c-int, F4/80-intmonocytic cells, inducing F4/80-hi, CD206-M1-like macrophages, consequently, enhances the immune activation response against apoptotic leukemia cells. In comparison with hNP/SnMP treatment, increased leukemic apoptosis in T-hNP/SnMP is a condition for immune boosting effect in HO1 checkpoint-inhibited myeloid cell.

8. Survival Study and Therapeutic Mechanisms of Chemo- and Immuno-Combination Therapy by HO1-Inhibiting T-hNP

Finally, in vivo therapeutic benefit with respect to survival was validated in an orthotopic AML model. A human AML-bearing xenograft model was injected with nanoparticles and DNR 6 times after cell infusion and its survival and body weight was monitored (FIG. 19a). The overall survival was 19, 24, 23, and 28 days for PBS, T-hNP +DNR, hNP/SnMP +DNR, and T-hNP/SnMP +DNR, respectively (FIG. 19b and FIG. c). Only moderate survival prolongation was demonstrated which is attributable to adaptive T cell immunity deficiency in NS G mice which is a key factor affecting cancer immunotherapy efficiency

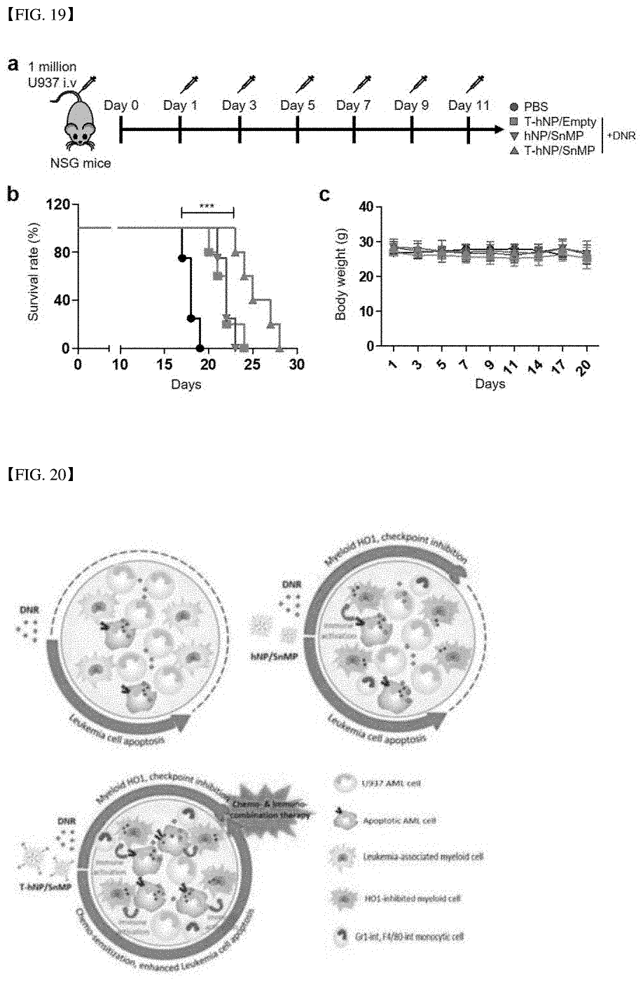

Collectively, the present inventors have developed T-hNPs/SnMP targeting HO1. Moreover, through DNR combination treatment, when the dual-targeting lipid-polymer hybrid nanoparticles (T-hNPs) of the present invention target cancer cells, the heme oxygenase 1 inhibitor acts to increase the reactivity of cancer cells to anti-cancer drugs (active targeting), but when targeting tumor-environment cells, the heme oxygenase 1 inhibitor acts to inhibit heme oxygenase 1, which acts as an anti-cancer immune checkpoint agent in macrophages within the tumor environment (passive targeting) (FIG. 20).

What is claimed is:

1. Dual-targeting lipid-polymer hybrid nanoparticles (T-hNPs) comprising a polymer core comprising a heme oxygenase 1 inhibitor, and a lipid membrane (shell) comprising a targeting moiety.

2. The nanoparticles according to claim 1, wherein the lipid constituting the lipid membrane is one or more selected from the group consisting of distearoylphosphatidylethanolamine (DSPE-PEG2000), 1,2-dilauroyl-sn-glycero-3-phosphocholine (DLPC), 1,2-dimyristoyl-sn-glycero-3-phosphocholine (DMPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-phosphocholine (DOPC), 1,2-dimyristoyl-sn-glycero-3-phosphoethanolamine (DMPE), 1,2-dipalmitoyl-sn-glycero-3-phosphoethanolamine (DPPE), 1,2-dioleoyl-sn-glycero-3-phosphoethanolamine (DOPE), 1,2-dimyristoyl-sn-glycero-3-phosphate (DMPA-Na), 1,2-dipalmitoyl-sn-glycero-3-phosphate (DPPA-Na), 1,2-dioleoyl-sn-glycero-3-phosphate (DOPA-Na), 1,2-dimyristoyl-sn glycero-3-phosphoglycerol (DMPG-Na), 1,2-dipalmitoyl-sn-glycero-3-phosphoglycerol (DPPG-Na), 1,2-dioleoyl-sn-glycero-3-phosphoglycerol (DOPG-Na), 1,2-dimyristoyl-sn-glycero-3-phosphoserine (DMPS-Na), 1,2-dipalmitoyl-sn-glycero-3-phosphoserine (DPPS-Na), 1,2-dioleoylsn-glycero-3-phosphoserine (DOPS-Na), 1,2-dioleoyl-sn-glycero-3-phosphoe thanolamine (DOPE-Glutaryl-(Na)2), Tetramyristoyl Cardiolipin-(Na)2, 1,2-distearoyl-sn-glycero-3-phosphoethanolamine (DSPE-mPEG-2000-Na), DSPE-mPEG-5000-Na, DSPE-Maleimide PEG-2000-Na, 1,2-dioleoyl-3-trimethylammonium-propane (DOTAP-Cl), and combinations thereof.

3. The nanoparticles according to claim 1, wherein the targeting moiety is immobilized to the lipid through a non-covalent bond.

4. The nanoparticles according to claim 3, wherein the non-covalent bond is a biotin/avidin or biotin/streptavidin bond.

5. The nanoparticles according to claim 4, wherein the biotin is bound to one end of the lipid, and the avidin or streptavidin is bound to one end of the targeting moiety.

6. The nanoparticles according to claim 1, wherein the heme oxygenase 1 inhibitor is one or more selected from the group consisting of SnMP, ZnMP, FeMP, MnMP, CrMP, SnPP, CrPP, and MnPP.

7. The nanoparticles according to claim 1, wherein the heme oxygenase 1 inhibitor is loaded into the polymer core at 3 to 7 w/w % based on the mass of the polymer.

8. The nanoparticles according to claim 1, wherein the polymer is one or more selected from the group consisting of poly(L-lactide) (PLLA), polyglycolic acid (PGA), polylactic acid (PLA), poly(lactic-co-glycolic acid) (PLGA), polycaprolactone (PCL), and poly(3-hydroxybutyrate-co-3-hydroxyvalerate) (PHBV), or a copolymer thereof.

9. The nanoparticles according to claim 1, wherein the lipid membrane and the polymer core are mixed so that a content of the lipid membrane is 0.15 to 0.35 w/w % based on the mass of the polymer core.

10. A method of preparing the dual-targeting lipid-polymer hybrid nanoparticles (T-hNPs) according to claim 1, the method comprising:
adding a heme oxygenase 1 inhibitor and a polymer mixture to an aqueous lipid solution in a dropwise manner and performing sonication to produce lipid-polymer hybrid nanoparticles; and
forming a targeting moiety on the lipid-polymer hybrid nanoparticles.

11. A kit for preparing the dual-targeting lipid-polymer hybrid nanoparticles (T-hNPs) according to claim 1, the kit comprising a container comprising a targeting moiety and a container comprising lipid-polymer hybrid nanoparticles.

12. A pharmaceutical composition comprising the dual-targeting lipid-polymer hybrid nanoparticles (T-hNPs) according to claim 1 as an active ingredient.

13. A method of preventing or treating cancer comprising administering the pharmaceutical composition according to claim 12 to a subject in need thereof.

14. The method according to claim 13, wherein dual-targeting lipid-polymer hybrid nanoparticles (T-hNPs) simultaneously target cancer cells and tumor-associated environmental cells.

15. The method according to claim 13, wherein in the treating of cancer, the pharmaceutical composition according to claim 12 is administered in combination with an anti-cancer agent.

16. The method according to claim 13, wherein the cancer comprises acute myeloid leukemia, bladder cancer, ovarian cancer, breast cancer, prostate cancer, melanoma, metastatic melanoma, lung cancer, non-small cell lung cancer, non-Hodgkin's lymphoma, hepatocellular carcinoma, brain cancer, glioma, or glioblastoma.
